(19) United States
(12) Patent Application Publication
(10) Pub. No.: US 2026/0232375 A1
(43) Pub. Date: Aug. 13, 2026

(54) SHAPE MEMORY ALLOY ENABLED ROBOTIC MODULAR MASS DEBRIDER

(71) Applicants: The Regents of the University of California, Oakland, CA (US); City of Hope, Duarte, CA (US)

(72) Inventors: Jun Sheng, Eastvale, CA (US); Behnam Badie, Duarte, CA (US)

(21) Appl. No.: 19/535,464

(22) Filed: Feb. 10, 2026

Related U.S. Application Data
(60) Provisional application No. 63/756,591, filed on Feb. 10, 2025.

(51) Int. Cl. A61B 18/14; A61B 17/00; A61B 17/32; A61B 17/3205; A61B 18/00; A61B 34/30

(52) U.S. Cl. CPC A61B 18/1492; A61B 34/30; A61B 2017/00867; A61B 2017/320032; A61B 2017/320064; A61B 17/32056; A61B 2018/00595; A61B 2018/1407; A61B 2034/301; A61B 2217/007

(57) ABSTRACT

A steerable apparatus for modular debriding is disclosed. The steerable apparatus can include: a shaft comprising a central lumen that extends along a length of the shaft; an adapter coupled to a proximal end of the shaft; at least one bearing comprising a central opening and positioned along the shaft such that the central opening is concentric with the central lumen and the adapter; at least one disk coupled to the shaft or to the at least one bearing; a pair of shape memory alloy (SMA) springs; a housing; a tip structure coupled to a distal end of the housing; and rotatable SMA cutting wire loops.

### CROSS-REFERENCE TO RELATED APPLICATION

This patent document claims priority to and benefits of U.S. Provisional Application 63/756,591, entitled “SHAPE MEMORY ALLOY ENABLED ROBOTIC MODULAR MASS DEBRIDER,” and filed on Feb. 10, 2025. The entire content of the above noted patent application is incorporated by reference as part of the disclosure of this patent document.

### STATEMENT REGARDING FEDERALLY SPONSORED RESEARCH

This invention was made with government support under R21 CA189223 awarded by the National Institutes of Health. The government has certain rights in the invention.

### TECHNICAL FIELD

This patent document relates to minimally invasive mass debulking techniques including steerable robotic tools.

### BACKGROUND

Some surgical procedures require removing mass from within an organ. In open surgery, surgeons open the organ to remove mass using tools such as dissectors and scissors. In minimally invasive surgery, surgeons make a small opening and insert a straight endoscopic tool equipped with channels for suction, irrigation, and drug delivery (such as blood thinner) into the organ to remove tissue. Both techniques present challenges: (i) open surgery involves a lot of risks and injury to patients, (ii) endoscopic suction does not work well for fibrous tissue, and (iii) endoscopic tools cannot remove large, irregularly shaped mass. Mass debulking techniques that address these challenges, among others, can provide positive surgical outcomes for patients.

### SUMMARY

The techniques described herein relate to a modular mass debrider that can be inserted into an organ through a small opening and a steerable robotic device to which the modular mass debrider can be connected. The modular debrider can be installed at the tip of a steerable surgical robot of any type including the steerable robotic device disclosed herein. By manipulating the steerable robotic device, the debrider can be steered inside the organ and used to remove mass at multiple locations to achieve conformal mass removal.

Methods, systems, and devices disclosed herein include a debrider apparatus comprising several cutting wires made of shape memory alloy. By electrically heating the cutting wires, the wires can be twisted to expand or shrink. In some implementations, a body of the debrider is a torsion actuator made of shape memory alloy springs. By electrically heating the springs, the cutting wires can rotate. Through the twisting and rotation of the cutting wires, mass can be fragmented and aspirated through the debrider. In some implementations, the debrider is also equipped with a channel for irrigation. The disclosed methods, systems, and devices also relate to a debrider tool installable on a steerable robotic device that can navigate inside an organ. Flexible shafts through the robotic device can be rotated to activate the debrider to fragment mass. In some implementations, suction and irrigation tubes through the robot can aspirate and irrigate the fragmented mass, respectively.

In one aspect, a steerable apparatus for modular debriding is disclosed. The steerable apparatus comprises: a shaft comprising a central lumen that extends along a length of the shaft; an adapter coupled to a proximal end of the shaft; at least one bearing comprising a central opening and positioned along the shaft such that the central opening is concentric with the central lumen and the adapter; at least one disk coupled to the shaft or to the at least one bearing; a pair of shape memory alloy (SMA) springs, the at least one disk or the at least one bearing being positioned along the shaft and between the pair of SMA springs such that the pair of SMA springs are spaced apart from one another along the length of the shaft by the at least one disk or the at least one bearing; a housing comprising (i) a top cover positioned at a top surface of the housing and (ii) a bottom cover positioned at a bottom surface of the housing, wherein the shaft, the central lumen, the at least one bearing, and the pair of SMA springs are disposed within the housing; a tip structure coupled to a distal end of the housing; and rotatable SMA cutting wire loops disposed on an external surface of the housing and coupled to the top cover and the tip structure.

In another aspect, a steerable apparatus for modular debriding is disclosed. The steerable apparatus comprises: a rigid tube; a precurved flexible tube disposed within the rigid tube and advanceable beyond a distal end of the rigid tube; a first flexible tube disposed within the precurved flexible tube and the rigid tube, a distal end of the first flexible tube extendable beyond a distal end of the precurved flexible tube; a second flexible tube disposed within the first flexible tube, the precurved flexible tube, and the rigid tube, a distal end of the second flexible tube extendable beyond the distal end of the precurved flexible tube and the distal end of the first flexible tube; and a tip structure coupled to the distal end of the second flexible tube and comprising rotatable SMA cutting wire loops, each of the rotatable SMA cutting wire loops disposed between a distal portion of the tip structure and an exterior surface of at least one of the distal end of the first flexible tube. In some implementations, the rigid tube, the precurved flexible tube, the first flexible tube, and the second flexible tube are coupled to one another so as to form a steerable tool operable to navigate the tip structure based on a direction of the precurved flexible tube and while the tip structure is located inside a mass. In some implementations, the direction of the precurved flexible tube is based on translating the rigid tube and translating and rotating the precurved flexible tube. In some implementations, the rotatable SMA cutting wire loops are configured to twist, expand, and rotate based on rotation of respective proximal ends of the first flexible tube and the second flexible tube with respect to one another.

In yet another aspect, a steerable apparatus for modular debriding is disclosed. The steerable apparatus comprises: a rigid tube; a tube comprising one or more notches located at a distal end of the tube; a straight flexible tube, disposed within the rigid tube and the tube, comprising a proximal end, wherein the straight flexible tube includes along its length one or more openings configured to align with the one or more notches; a tip structure located at the distal end of the tube and configured to cap the straight flexible tube at one end; and metallic wires forming loops disposed between the tip structure and the tube, wherein the rigid tube, the tube, and the straight flexible tube are coupled to one another so as to form a steerable tool operable to navigate the tip structure, wherein rotation of the proximal end allows the metallic wires to be twisted and expanded.

### BRIEF DESCRIPTION OF THE DRAWINGS

FIG. 1 shows an example of a mass debrider tool according to an embodiment of the technology disclosed in this patent document.

FIG. 2A shows a schematic of an example part of a mass debrider tool according to an embodiment of the technology disclosed in this patent document.

FIG. 2B shows a schematic of an example part of a mass debrider tool according to an embodiment of the technology disclosed in this patent document.

FIG. 2C shows a schematic of an example part of a mass debrider tool according to an embodiment of the technology disclosed in this patent document.

FIG. 2D shows a schematic of an example part of a mass debrider tool according to an embodiment of the technology disclosed in this patent document.

FIG. 3A shows a schematic of an example component of a mass debrider tool according to an embodiment of the technology disclosed in this patent document.

FIG. 3B shows a schematic of an example component of a mass debrider tool according to an embodiment of the technology disclosed in this patent document.

FIG. 4 shows an example of a debriding apparatus according to an embodiment of the technology disclosed in this patent document.

FIG. 5 shows an example component of a debriding apparatus according to an embodiment of the technology disclosed in this patent document.

FIG. 6 shows an example component of a debriding apparatus according to an embodiment of the technology disclosed in this patent document.

FIG. 7 shows another example of a debriding apparatus according to an embodiment of the technology disclosed in this patent document.

FIG. 8 shows a schematic of a working principle of an example debriding apparatus according to an embodiment of the technology disclosed in this patent document.

FIG. 9A shows an image of an example debriding apparatus according to an embodiment of the technology disclosed in this patent document.

FIG. 9B shows an image of an example debriding apparatus according to an embodiment of the technology disclosed in this patent document.

FIG. 10 shows a series of images illustrating an example fabrication method for a debriding apparatus according to an embodiment of the technology disclosed in this patent document.

FIG. 11 shows images of an example integrated robotic system including an actuation system and a steerable robot according to an embodiment of the technology disclosed in this patent document.

FIG. 12 shows an example schematic defining local coordinate frames on a robot body according to an embodiment of the technology disclosed in this patent document.

FIG. 13A shows an experimental setup for a motion control test of an example device according to an embodiment of the technology disclosed in this patent document.

FIG. 13B shows images of an example robot moving to three desired target positions in the motion control test of FIG. 13A.

FIG. 14 shows an example data plot of measured positions of a robot tip that were obtained in an example study performed in accordance with disclosed techniques.

FIG. 15 shows an image of an example experimental setup that can be used to demonstrate the ability of some example robots to remove brain mass lesions in accordance with some example embodiments.

FIGS. 16A-16L shows images of tissue removal that can be performed using an example robotic system according to an embodiment of the technology disclosed in this patent document.

FIG. 17 shows an example data plot obtained in a study performed in accordance with disclosed techniques.

FIG. 18 shows a schematic of an example implementation of a steerable robotic device according to an embodiment of the disclosed technology

### DETAILED DESCRIPTION

Brain mass lesions are a broad collection of pathological processes that result in changes inside the brain and include conditions such as infection, hemorrhage, and brain tumors. Due to their presence inside one of the most critical and delicate organs, their impact on patient health is significant regardless of the types of mass lesions. For example, spontaneous intracerebral hemorrhages (ICH) affects nearly 67,000 patients every year in the US. Malignant brain tumors are another type of life-threatening brain mass lesion.

Minimally invasive surgery is regarded as a safer approach than open craniotomy to remove deep intracerebral mass lesions such as hematomas. It is usually performed by introducing a straight suction tool, sometimes combined with accessories for tissue debridement and irrigation, into the brain. Since the collateral trauma to healthy tissue is proportional to the diameter of the tools, slender tools with small diameters are desired. However, current minimally invasive tools are inadequate for removal of large, multi-focal, and fibrous mass lesions.

For many types of brain mass lesions, such as stable ICHs and brain tumors, debulking remains the gold standard procedure to save patients' lives and alleviate symptoms caused by raised intracranial pressure. In the past decade, minimally invasive surgery (MIS) techniques have emerged to improve surgical outcomes by reducing collateral brain injury caused by standard craniotomy. MIS techniques allow neurosurgeons to access brain mass lesions by inserting a cannula through a burr hole and a sulcus along a trajectory that runs parallel to the white matter tracts. Soft mass lesions (such as hematomas) can be evacuated under direct or endoscopic visualization using a tissue debrider (e.g., NICO Myriad® System) or a suction device (e.g., Penumbra Artemis™ Neuro Evacuation Device) through the cannula. Although less invasive than standard craniotomy, these MIS approaches sometimes fail to achieve an acceptable balance between the extent of removal and neural tissue disruption. When accessing deeply located, large lesions, the large diameter of the cannulas and pivoting and redirecting the cannula shaft could cause white matter disruption. In addition, efficiency of the procedure is low, which limits their application in urgent cases, such as unstable ICHs. For example, some have reported that the average duration to reach an acceptable residual ICH size was 3.6 days, while the ideal time should be less than 24 hours of ictus.

Robots have been developed to improve the precision ofneurosurgery (e.g., Rosa One® Brain, Remebot®, and CAS-R-2®). However, these robots only guide the placement of tools, and the lesion removal procedure is performed using standard or MIS techniques. To improve the safety and efficacy of neurosurgery, a few steerable mesoscale robots have been developed for manipulation inside brain mass lesions. Concentric tube robots (CTRs) are popular due to their small footprints and high dexterity. A CTR typically consists of thin, precurved, elastic tubes nested within one another and can steer its tip by translating and rotating individual tubes. CTRs consisting of a straight tube and a precurved tube have been developed for ICH removal. The present patent document discloses various embodiments which can achieve a larger internal channel and integrate a tissue cutting tool to enhance tissue removal. A MEMS-based tissue cutting tool delivered via CTRs was developed for precise tissue removal in the beating heart, but it may not be suitable for efficient debulking of brain mass lesions. Another type of steerable neurosurgical robots is the tendon-driven robot (TDR) which typically consists of an elastic continuum body and tendon wires routed from outside to the robot body and steers its tip through tendon displacement. So far, magnetic resonance-conditional TDRs have also been developed for brain tumor removal. TDRs offer higher force capabilities; how-ever, the complexity of tendon routing in TDRs can introduce challenges in terms of robot design and control systems. A hybrid design combining the CTR and TDR mechanisms was developed for ICH evacuation, allowing enhanced versatility in navigating confined spaces while delivering larger force for tissue manipulation. In addition, shape memory alloy (SMA) robots featuring discrete SMA joints were also developed for ICH removal. Although their function have been demonstrated using soft gelatin and aspiration, their efficacy for brain mass lesions is yet unknown.

In one aspect, the present patent document provides a robotic surgical device for removing intracerebral mass lesions. An example device can include four concentric tubes including a straight rigid stainless steel tube, a precurved superelastic nitinol tube with asymmetric notches, a braid-reinforced composite tube with tissue cutting holes at the tip, and a suction tube connected with a suction machine. From outermost to innermost, the tubes may be configured as: straight rigid stainless steel tube, precurved superelastic nitinol tube with asymmetric notches, braid-reinforced composite tube with tissue cutting holes at the tip, and suction tube connected with a suction machine. In some embodiments, a Pebax sleeve covers the notched area of the outer tube except the two most distal notches. By rotating and translating the notched nitinol tube, the robot tip can be manipulated inside a mass lesion. By concurrently rotating the cutting tube and applying negative pressure, tissues can be cut and removed through the suction tube.

In another aspect, the present patent document provides design and fabrication techniques for various embodiments of robotic devices based on the disclosed technology, in addition to kinematic modeling of some robots in terms of the rotation and translation of the notched tube and rotation of the cutting tube. Results of an example study, performed in accordance with disclosed techniques, show 540.68% improvement of mass lesion removal efficiency using an example device based on the disclosed technology.

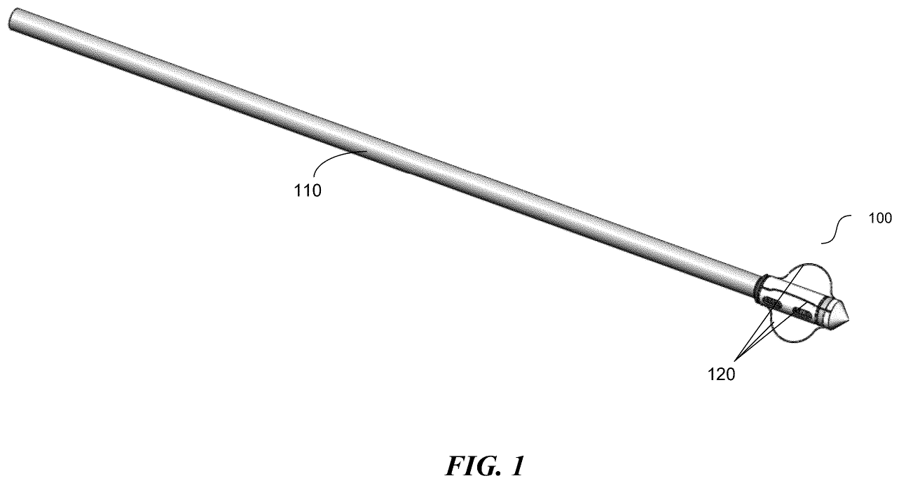

FIG. 1 shows an example embodiment of a modular debrider apparatus 100. The modular debrider 100 can be installed at the tip of a surgical tool such as a surgical tool comprising a straight rigid tube 110. In some implementations, the surgical tool may include or be coupled to a steerable robot. In some implementations, the modular debrider apparatus 100 is coupled to a steerable robot. The modular debrider 100 is equipped with cutting wires 120 that can expand, shrink, and rotate to fragment mass, an irrigation channel, and a suction channel. Therefore, tissue mass can be easily debulked.

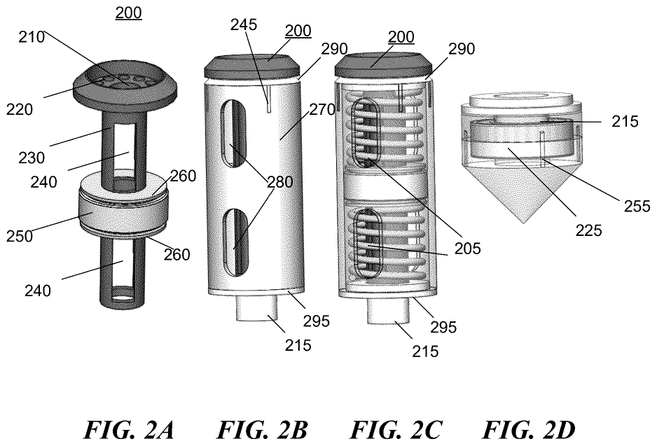

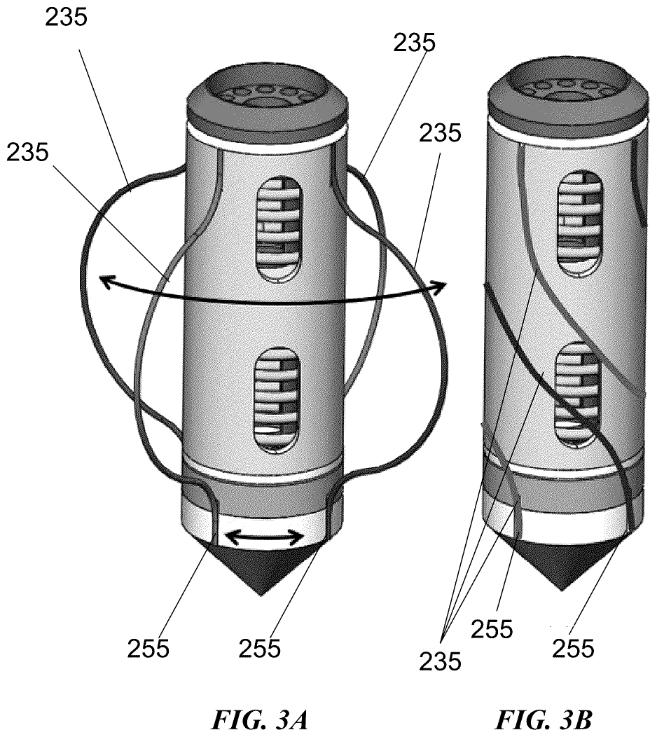

In some embodiments, a modular debrider apparatus (e.g., 100) includes an adapter 200 (FIG. 2A) that can be combined with the tip of a slender surgical tool. The adapter 200 includes a hole 210 in the center for a suction channel and electric wires as well as several small holes 220 around the center for irrigation. The adapter 200 is combined with a main shaft 230 which has openings 240 along the length as shown in FIG. 2A. A bearing 250 is installed in the middle of the shaft 230 and covered by two disks 260 fixed, respectively, to two sides of the inner race of the bearing 250. The bearing 250 supports the rotation of a casing 270 comprising a cylindrical structure with openings 280, a top cover 290, and a bottom cover 295 (FIG. 2B). As shown in FIG. 2C, two shape memory alloy (SMA) springs 205 are fixed between the bearing disks 260 and covers 290, 295. The SMA springs 205 are tightened during assembly and the bottom casing cover 295 includes a short hollow shaft 215. A bearing 225 is installed on the shaft 215 and combined with two tip covers (FIG. 2D). Both the casing 270 and tip covers have small evenly spaced grooves. As shown in in FIG. 3A, SMA wires 235 which are trained to memorize a helical shape are fixed inside the grooves 245 and 255 located at two opposite ends of the debrider (FIGS. 2A-2C). The memorized helical shapes of adjacent SMA wires 235 are opposite. Electrical wires pass through the shafts 230, 215 and are connected to the SMA wires 235 and springs 205.

In some embodiments, by heating and cooling the two SMA springs 205 alternately, the casing 270 and tip covers rotate around the main shaft 230 bidirectionally, and thereby rotate the SMA wires 235. In some embodiments, by heating and cooling the two groups of SMA wires 235 alternately, the SMA wires 235 twist bidirectionally, with the tip covers rotating around the casing 270 as shown in FIG. 3B. Mass fragmented by the twisting and rotating SMA wires 235 will be aspirated into the casing, further fragmented by SMA springs 205, and finally aspirated and debrided through the main shaft 230 and suction channel 210.

Since mass can be fragmented by the rotating twisted cutting wires 235, it can be efficiently and effectively removed. By navigating the modular debrider to different locations inside the organ using a steerable robot, large and irregular shaped mass can be removed. Thus, the modular apparatus provides an effective and safe method to remove mass from within an organ.

In another example embodiment, a modular debrider apparatus includes at least one bearing installed at a proximal and/or distal end of the shaft. The outer race of the at least one bearing is coupled to the top and/or bottom cover of the casing. A single disk is coupled to the middle of the shaft. Two SMA springs are fixed between the single disk and the covers.

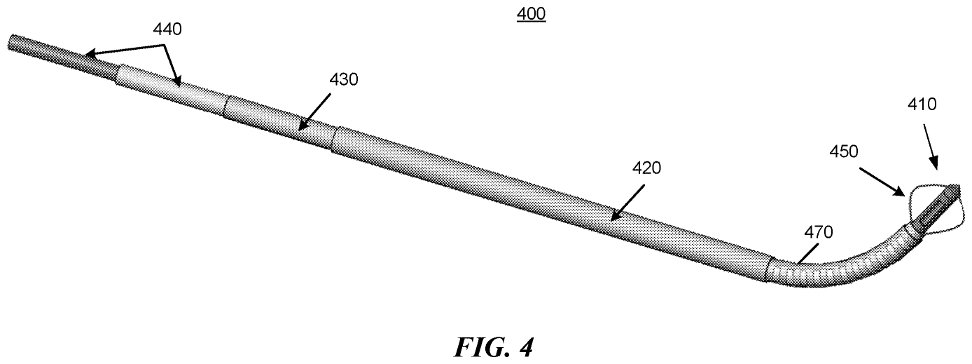

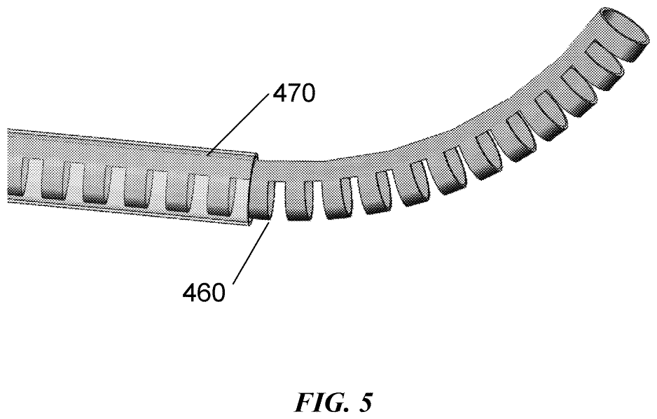

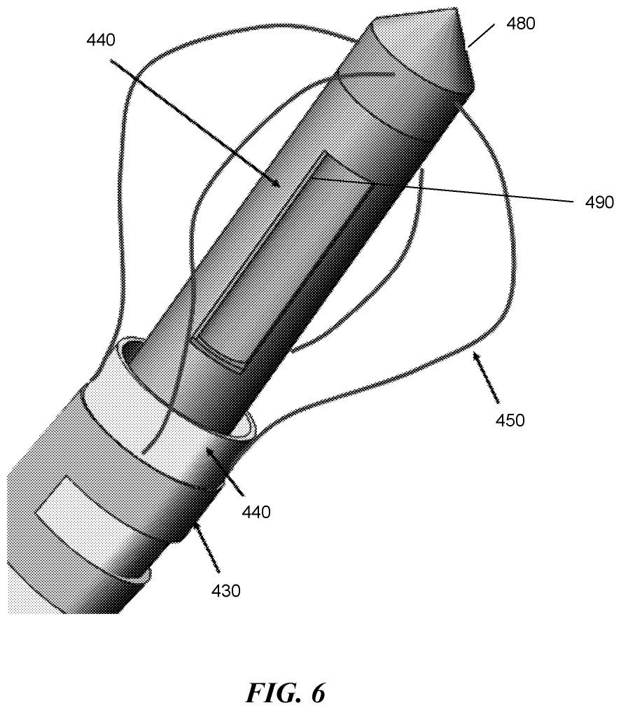

FIG. 4 shows an example embodiment of a steerable debriding apparatus 400. One unique feature of the apparatus 400 is the ability to steer a mesoscale debrider 410 located at the tip of the steerable debriding apparatus 400 inside soft tissue to debulk mass in different locations. The debrider 410 includes cutting wires 450. As shown in FIG. 4, the steerable debriding apparatus 400 comprises a straight rigid tube 420, a precurved flexible tube 430, and two straight flexible tubes 440. The straight rigid tube 420, precurved flexible tube 430, and two straight flexible tubes 440 are assembled in a telescopic manner such that the straight flexible tubes 440 are passively curved. The two straight flexible tubes 440 are located inside the rigid tube 420 and the precurved flexible tube 430. A compliant sleeve 470 covers the precurved flexible tube 430. As shown in FIG. 5, the precurved flexible tube 430 is made of a superelastic SMA tube with asymmetric notches 460 along the outer bending edge and a compliant sleeve 470 covers the precurved flexible tube 430. As shown in FIG. 6, the tips of the two straight flexible tubes 440 may extend beyond the tip of the precurved tube 430 and are located apart from each other. A conic tip 480 with a shaft 490 is used to cap the front end of the inner straight flexible tube 440. Both the inner straight flexible tube 440 and the shaft 490 have at least one side opening at a distal side of the steerable debriding apparatus 400. The apparatus 400 also includes several metallic wires 450 which form loops between the tips of the two straight flexible tubes 440. In some implementations, the inner and outer straight flexible tubes 440 are connected to a suction machine and irrigation machine, respectively.

By translating the rigid tube 420 and translating and rotating the precurved flexible tube 430, the tip of the flexible tube 430 can be steered to navigate the wire loops to different locations. By rotating the proximal ends of the two straight flexible shafts 440, the wire loops 450 can be twisted, expanded and rotated. Meanwhile, irrigation can be provided through the gap between the two straight flexible shafts 440, and suction can be performed through at least one side opening of the inner straight flexible tube 440. Electric current can be applied through electric wires between the precurved tube 430 and the outer straight flexible tube 440 to the wire loops 450 for unipolar electrocauterization.

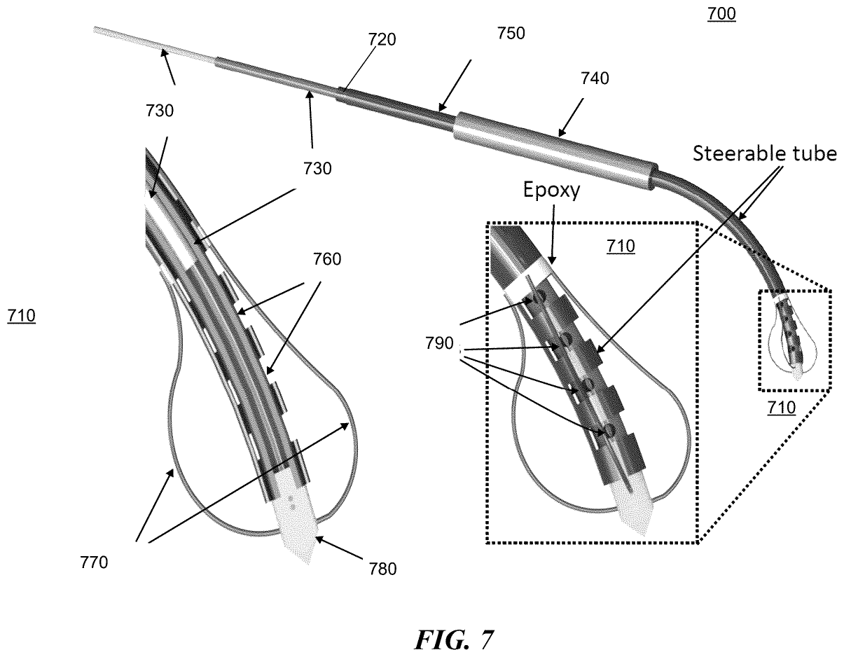

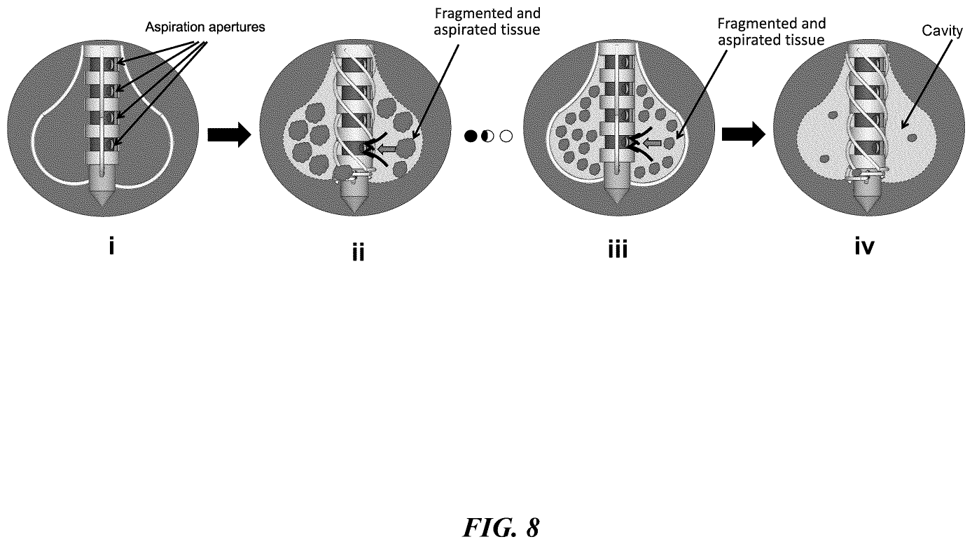

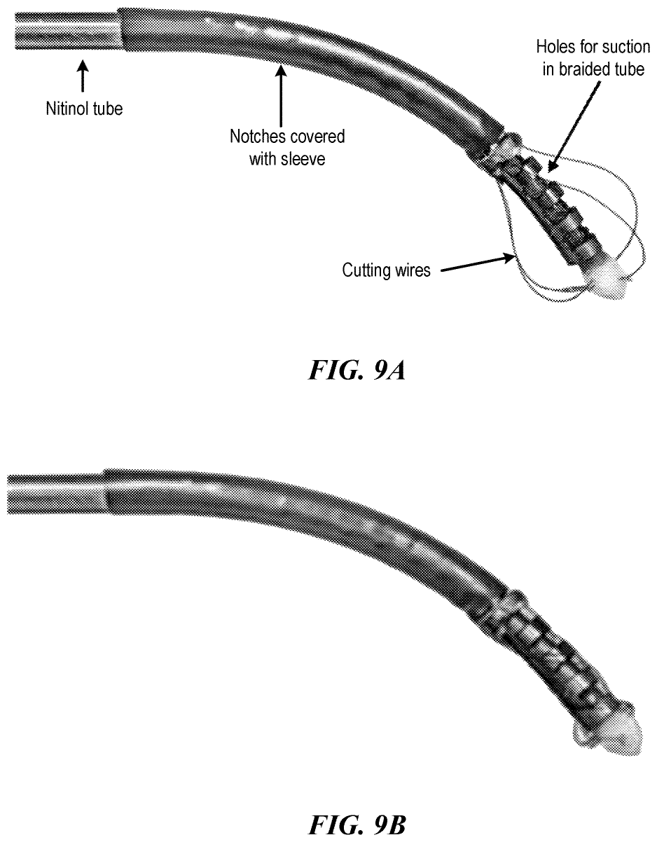

FIG. 7 (top) shows an isometric view of an example embodiment of a steerable robotic debriding apparatus 700, with the inset of FIG. 7 showing details of the robot tip 710 equipped with a tissue cutting and aspiration mechanism. FIG. 7 (lower left) shows a section view of the robot tip 710, illustrating the concentric configuration of the precurved tube 720 and two polymer tubes 730. As shown in FIG. 7, the main body of the steerable robot 700 is a concentric tube robot comprising a straight rigid outer tube 740 and a precurved elastic inner tube 720 with notches 760 along the length. By translating these two tubes 740 and 720 and rotating the precurved tube 720, the robot tip 780 can be manipulated in 3D space to cover desired workspace. By holding the outer tube 740 still and translating the precurved tube 720, the robot can advance along a constant-curvature trajectory and follow the leader. In the example of FIG. 7, a sleeve 750 (e.g., Pebax sleeve) covers the notches 760 of the precurved tube 720 to reduce the friction between the two tubes. In some implementations, inside the precurved tube 720, there are two off-the-shelf braid-reinforced polymer tubes which may be made of, e.g., jacket and Pebax liner. In some implementations, both polymer tubes 730 rotate and translate concurrently with the precurved tube 720. The tip of the outer polymer tube 730 is aligned with the precurved tube 720, and a conic tip 780 is mounted at the tip of the outer polymer tube 730. At the distal end of the outer polymer tube 730, four 1.50 mm diameter aspiration apertures 790 are drilled along the length such to align each aperture 790 with a notch 760 of the precurved tube 720 in the axial direction. The tip of the inner polymer tube 730 stops before the most proximal aspiration aperture, and the inner polymer tube 730 can be connected to a medical aspiration device. Four 0.15 mm diameter superelastic nitinol wires can be installed at the robot tip 90-degree apart as tissue cutting wires 770. One end of each wire 770 can be fixed in the hole of the conic tip 780 and the other end on the outer surface of the precurved tube 720 prior to the most proximal aspiration aperture using epoxy. By rotating the outer polymer tube relative 730 to the precurved tube 720, the four nitinol wires 770 can be twisted to expand or collapse, resulting in the fragmentation of the tissue in the envelope of the cutting wires as shown in FIG. 8. Meanwhile, by supplying suction pressure through the inner polymer tube 730, fragmented tissue can be drawn into the aspiration apertures 790 when the aspiration apertures face towards the notches 760, dissected by the edges of the apertures, and finally removed through the inner polymer tube 730. FIG. 9A and FIG. 9B show the tip of an example surgical robot device with fully expanded and collapsed wire loops, respectively.

As shown in FIG. 7, the steerable, robotic debriding apparatus includes one straight flexible tube 730 located inside a rigid tube 740 and a precurved flexible tube 720. A compliant sleeve 750 partially covers the precurved flexible tube 720 such that notches 760 at the distal end of the precurved flexible tube 720 are exposed. A conic tip 770 with a shaft is used to cap the front end of the straight flexible tube 730. The straight flexible tube 730 includes openings (e.g., suction holes) 790 along its length that are aligned with the exposed notches 760 of the precurved flexible tube 720. Several metallic wires 770 form loops between the conic tip 780 and the precurved flexible tube 720 near the first exposed notch. By rotating the proximal end of the straight flexible tube 730, the wire loops 770 can be twisted/expanded. In some implementations, the precurved flexible tube 720 comprises a superelastic SMA material. In some implementations, the flexible tube 720 comprising the superelastic SMA material is not precurved and is instead a straight tube with notches along its length. In some implementations, at least one superelastic SMA wire is attached to the tip of the flexible tube 720 comprising the superelastic SMA material and routed through the straight flexible tube 730 to actuators located external to the steerable debriding apparatus 700. By translating the rigid tube 740 and the precurved flexible tube 720 and pulling tendon wires, the tip of the precurved flexible tube 720 can be steered to navigate the wire loops 770 to different locations.

In some implementations, a modular debrider apparatus (e.g., 100) is installed at a distal end of the steerable debriding apparatus (e.g., 700). By navigating the modular debrider to different locations inside an organ, the steerable debriding apparatus can remove large and irregular shaped masses, thereby expanding the function of the debrider and providing an effective and safe method to remove masses from within an organ.

Example dimensions for the outer diameter (OD) and inner diameter (ID) of some example tubes in accordance with some disclosed embodiments are summarized in Table I below.

TABLE I
Example geometric specifications of a robot (unit: mm)

| Variable | Physical Meaning | Value |
|---|---|---|
| dₒ_{ₛₜ} | Outer diameter of the straight tube | 9.62 |
| dᵢ_{ₛₜ} | Inner diameter of the straight tube | 6.06 |
| dₒ_{ₛₗᵥ} | Outer diameter of the sleeve | 5.68 |
| dᵢ_{ₛₗᵥ} | Inner diameter of the sleeve | 5.23 |
| dₒ_{ₚₜ} | Outer diameter of the precurved tube | 5.10 |
| dᵢ_{ₚₜ} | Inner diameter of the precurved tube | 4.7 |
| dₒ_{ₒₚ} | Outer diameter of the outer polymer tube | 3.63 |
| dᵢ_{ₒₚ} | Inner diameter of the outer polymer tube | 2.74 |
| dₒ_{ᵢₚ} | Outer diameter of the inner polymer tube | 2.26 |
| dᵢ_{ᵢₚ} | Inner diameter of the inner polymer tube | 1.77 |
| L_{w} | width of precurved tube notch | 2.00 |
| L_{d} | depth of precurved tube notch | 4.26 |
| Lₛ | spacing of precurved tube notch | 2.00 |
| dₐₛₚ | diameter of aspiration apertures | 1.5 |
| Lₐₛₚ | spacing of aspiration apertures | 5.22 |

FIG. 8 shows a schematic of a working principle of an example surgical robotic device (e.g., 700). At step (i), wire loops are deployed and unwound in the mass lesion. At step (ii), tissue is fragmented by alternately winding and unwinding wire loops and removed by aspiration. At step (iii), tissue is further fragmented and removed by continuous wire loop operation and aspiration. At step (iv), a cavity is generated at the end of operation.

Traditional concentric tube robots made of superelastic nitinol tubes with small diameter can be manipulated in tissue with minimal damage to tissue structures due to their small footprints. This is one rationale of applying them for debulking of brain mass lesions. However, the small channel through the nitinol tubes may prevent the robot from effectively removing tissue, especially when the tissue is firm and fibrous. To achieve a better balance between safety and effectiveness and enable the incorporation of polymer tubes, some disclosed embodiments provide a concentric tube robot with a larger channel. It is noted that scaling up the design of concentric tube robots is nontrivial. Small-diameter, thin-wall nitinol tubes can be used to build a sharply curved robot. However, due to the limited recovery strain of approximately 10%, large-diameter Nitinol tubes, which typically have thick walls, are challenging to use in constructing large-channel robots with sufficient precurvature.

Some disclosed embodiments can address the foregoing challenges. For example, in some example devices the outer tube is a 3D-printed plastic tube comprising VeroWhite resin material.

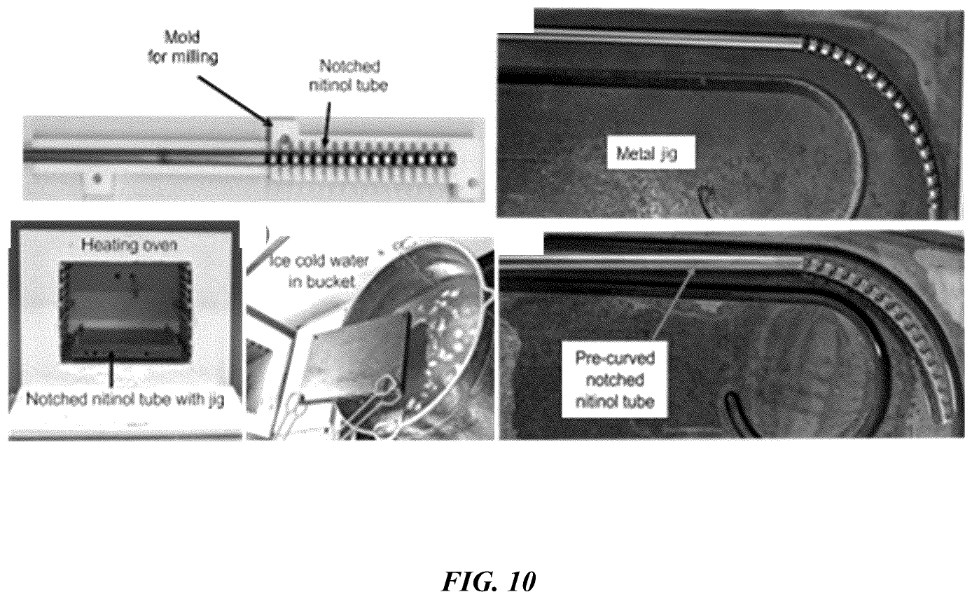

FIG. 10 shows a series of images illustrating a fabrication method for a steerable robot based on the disclosed technology. In an example fabrication process of a flexible, large-channel precurved elastic tube bypassing the strain limit, the following steps were performed on a 5.10 mm OD and 4.70 mm ID superelastic nitinol tube: milling discrete notches along a straight line at the distal end of the nitinol tube in a straight 3D-printed plastic mold using a desktop milling machine—the width, depth, and spacing of notches were 2 mm, 4.26 mm, and 2 mm, respectively (FIG. 10, top left); fitting the notched nitinol tube inside the milled curved groove of a steel block—the notches of the tube face outside for smooth translation of the curved notched tube against the outer tube (FIG. 10, top left); heating the nitinol tube fixed in the block at 550° C. for 30 minutes (FIG. 10, lower left); quenching the tube and block assembly inside room temperature water for about 1 minute (FIG. 10, lower middle); and removing the nitinol tube from the block (FIG. 10, lower right). The curvature of the fabricated tube was 13.51 m⁻¹. Since the notches shift the neutral bending plane from the center of the tube to the remaining wall, the tube is compliant to bend in the notched areas but maintain its rigidity between the notches. Therefore, during shape setting, the entire tube can conform to the curved shape of the groove under the strain limit due to the deflection of the notched areas of the tube. For the same reason, when the thermally trained, precurved nitinol tube is assembled with the straight outer tube, it can conform to the shape of the outer tube under the strain limit during translation.

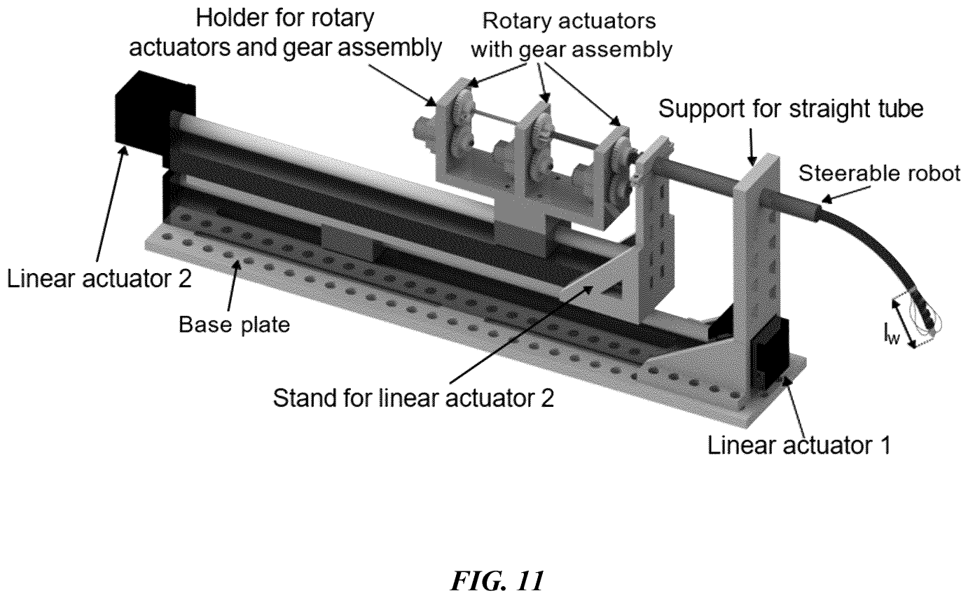

FIG. 11 shows a schematic of an example integrated robotic system including an actuation system and a steerable robot (e.g., 700) with a tissue cutting and aspiration mechanism. The integrated robotic system can include a holder for rotary actuators and gear assembly, rotary actuators with gear assembly, a support for a straight tube, a steerable robot, linear actuators, a base plate, and a stand for a linear actuator. The actuation system can include some or all of the following functions: 1) linearly moving the steerable robot to insert the robot into or retract the robot from a brain mass lesion through a burr hole; linearly moving and rotating the precurved tube together with the two polymer tubes with respect to the outer tube to navigate the mass lesion; rotating the two polymer tubes with respect to the precurved tube and outer tube to expand and collapse the cutting wires.

In one example, the main structure of the actuation system was built by stacking a short (250 mm stroke) linear motion stage on a long (300 mm stroke) one mounted on a base plate. Both stages are off-the-shelf (Zeberoxyz, China) and made of a stepper motor coupled with a lead screw and a linear guide rail. On the guide rail carriage of the long motion stage, both the guide rail base of the short motion stage and the base of the outer tube are fixed using 3D-printed plastic connectors. The bases of the precurved tube and two polymer tubes are installed on the guide rail carriage of the short motion stage through three rotation units for the inner polymer tube, outer polymer tube, and precurved tube from the proximal to the distal end, respectively. Each rotation unit is fixed to the carriage and hosts a ball bearing and a rotary DC motor (155 rpm@6V, 100:1 gear reduction, UXCell, Hong Kong, China) with a spur gear on the shaft. By fixing the base of the precurved or polymer tube inside the bearing and engaging the tube with the motor shaft through gear meshing, the tube can be rotated by the motor. To support the robot to move linearly, a vertical frame with a hole is fixed on the base plate at the distal end of the long linear motion stage and the entire robot goes through the supporting hole.

To control the robotic system of FIG. 11, L298N and TB6600 drivers are used for driving the DC motors and stepper motors, respectively, and a Model 826 analog and digital I/O system (Sensoray, Portland, OR USA) was used to communicate between a control program based on Visual Studio C++ and the drivers and DC motor encoders. Pulse width modulation was used for position control of the stepper motors. The rotary position of the DC motors was feedback controlled based on the encoder reading using a proportional-integral controller.

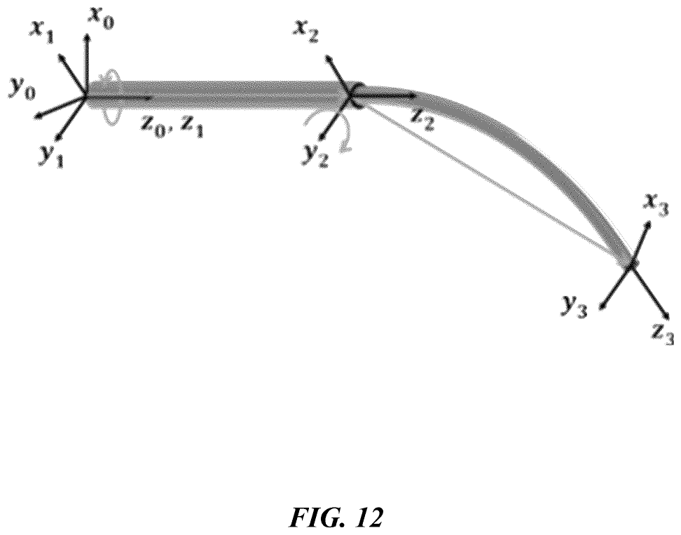

In another aspect, a kinematic model was developed to map the actuator space q into the task space x in accordance with some disclosed embodiments. FIG. 12 shows an example schematic defining local coordinate frames on a robot body for kinematic modeling. In some example embodiments, since the polymer and precurved tubes move together, the variables in the q space include the translation (sᵢ) and rotation (θᵢ) of the precurved tube and translation (sₒ) of the outer tube, namely, q={sᵢ, θᵢ, sₒ}. Assuming the tips of the outer tube and precurved tube are aligned in the home positions, by extending the precurved tube out of the outer tube, the length of curved part of the robot is given by: l_{c}, =sᵢ−sₒ, and the length of the straight part is given by iₛ=sₒ. To deploy the tissue cutting and aspiration mechanism, the precurved tube needs to be extended out of the outer tube by at least l_{w}. Hence, one has l_{w}<l_{c}<lₙ, where lₙ is the axial length of the notched part of the precurved tube. Although the curved part of the robot is made of serially connected, alternate curved (notched areas) and straight (areas between notches) segments, the curvature of the curved segments is constant along the length of each segment and the same across the segments, and a global curvature (k_{c}), which is assumed to be constant along the length of the curved part of the robot, is used to describe the configuration of the robot. The value of k_{c} was estimated by outlining the notched part of the precurved tube covered by the sleeve and hosting the polymer tubes on the paper, finding the centroid, and measuring the radius. Due to the creep effect of the polymer tubes inside the precurved tube, their precuratures are not zero, and the value of k_{c} will be different when the precurved tube and the polymer tubes rotate relatively. Due to the lower stiffness and smaller precurvutures of the polymer tubes compared to the precurved tube, the variation of k_{c} will be small. To simplify robot control, the polymer tubes are reset to the initial rotary position after tissue removal, assuming that the creep effect would not change during tissue removal. Therefore, the same value of K, can be used for control throughout the entire process.

To model the robot kinematics, a global coordinate frame, {F₀}, is attached to the home position with z₀-axis pointing to the extension direction of the straight tube and x₀-axis in the bending plane of the precurved tube when θᵢ=0, as shown in FIG. 11. Three local coordinate frames are defined along the robot length. {F₁} is defined at the home position with a rotational offset from {F₀} around z₀ by θᵢ. {F₂} is defined at the tip of the outer tube with a translational offset from {F₁} along z₁ by sₒ. {F₃} is defined at the tip of the precurved tube with the z₃-axis in the tangential direction along the robot and y₃-axis aligned with y₂-axis. Based on these definitions, the transformation between them is given by:

$$\begin{matrix}{T}_{0}^{1} = \begin{matrix}- cos ( {θ}_{i} ) & - sin ( {θ}_{i} ) & 0 & 0 \\ sin ( {θ}_{i} ) & cos ( {θ}_{i} ) & 0 & 0 \\ 0 & 0 & 1 & 0 \\ 0 & 0 & 0 & 1\end{matrix} , {T}^{2} = \begin{matrix}1 & 0 & 0 & 0 \\ 0 & 1 & 0 & 0 \\ 0 & 0 & 1 & {s}_{o} \\ 0 & 0 & 0 & 1\end{matrix} , \\ {T}_{2}^{3} = \begin{matrix}cos ( {κ}_{c} ( {s}_{i} - {s}_{o} ) ) & 0 & sin ( {κ}_{c} ( {s}_{i} - {s}_{o} ) ) & \frac{1 - cos ( {κ}_{c} ( {s}_{i} - {s}_{o} ) )}{{κ}_{c}} \\ 0 & 1 & 0 & 0 \\ - sin ( {κ}_{c} ( {s}_{i} - {s}_{o} ) ) & 0 & cos ( {κ}_{c} ( {s}_{i} - {s}_{o} ) ) & \frac{sin ( {κ}_{c} ( {s}_{i} - {s}_{o} ) )}{{κ}_{c}} \\ 0 & 0 & 0 & 1\end{matrix}\end{matrix}$$

The position of the robot tip can be modeled by concatenating the above transformation matrices as

$${T}_{0}^{3} = {T}_{0}^{1} {T}_{1}^{2} {T}_{2}^{3}$$

and examining the last column of

$${T}_{0}^{3} ,$$

which yields:

$$x = \frac{cos ( {θ}_{i} ) ( 1 - cos ( ( {s}_{i} - {s}_{o} ) {κ}_{c} ) )}{{κ}_{c}} \qquad ( 1 )$$

$$u = \frac{sin ( {θ}_{i} ) ( 1 - cos ( ( {s}_{i} - {s}_{o} ) {κ}_{c} ) )}{{κ}_{c}} \qquad ( 2 )$$

$$z = {s}_{o} + \frac{( sin ( ( {s}_{i} - {s}_{o} ) {κ}_{c} ) )}{{κ}_{c}} \qquad ( 3 )$$

To solve the inverse kinematics, Eqs. (1) and (2) are combined, yielding:

$${θ}_{i} = a tan 2 ( y , x ) \qquad ( 4 )$$

With the computed θᵢ, sₒ is solved by combining Eqs. (1) and (3) as follow:

$${s}_{o} = z - … \frac{x}{{κ}_{c} cos ( {θ}_{i} )} ( 2 - \frac{x {κ}_{c}}{cos ( {θ}_{i} )} ) \qquad ( 5 )$$

With the computed θᵢ and sₒ, sᵢ is solved by reusing Eqs. (1) and (3) as:

$${s}_{i} = {s}_{o} + \frac{a tan 2 ( {κ}_{c} ( z - {s}_{o} ) , 1 - \frac{x κ c}{cos ( {θ}_{i} )} )}{{κ}_{c}} \qquad ( 6 )$$

The inverse kinematics Eqs. (4) to (6) allow one to calculate the required values of q space variables and control the robot motion for desired values of x, y, and z.

Various example studies were performed to characterize some example robot devices based on the disclosed technology.

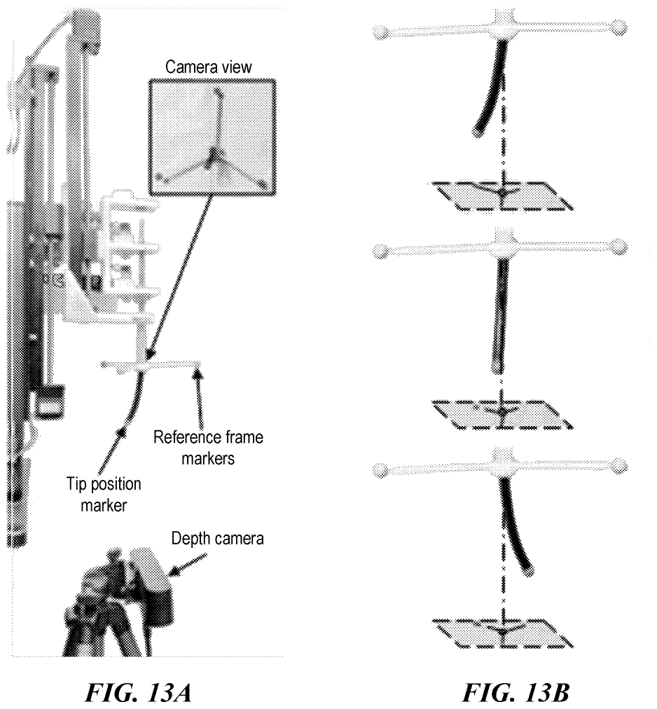

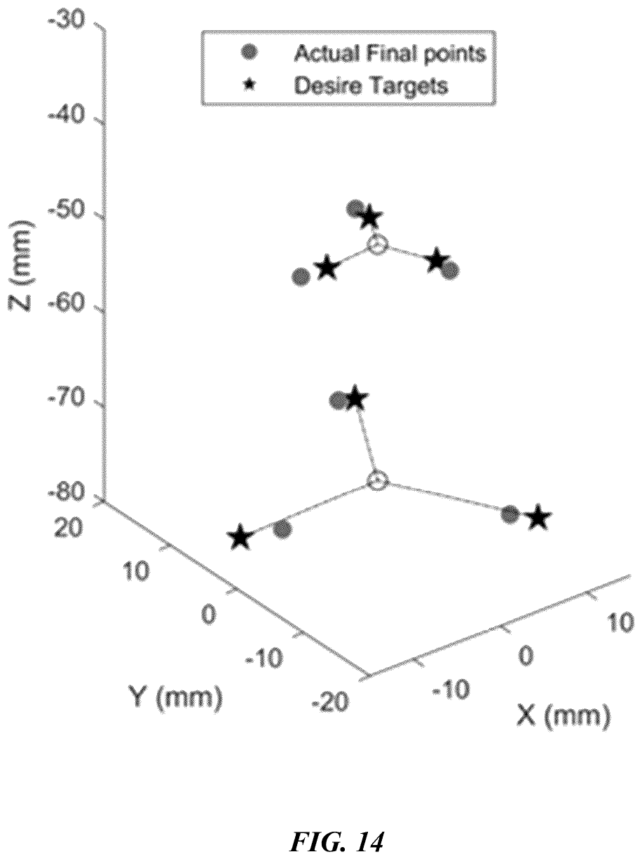

To verify ability of an example robot to navigate to individual target positions inside a lesion, an experimental study was performed to evaluate the tip position error. FIG. 13A shows an experimental setup for the motion control tests with the inset showing a camera view. In this study, the outer tube was fixed vertically, and the precurved tube was initially retracted into the outer tube and then manipulated to access different target positions. Six target positions that required zero translation of the outer tube were designed at two different heights in three 120°-apart planes in this study. FIG. 13B shows snapshots of the robot moving to three desired target position at the same height. The corresponding reference trajectories were computed based on the kinematic model. Based on the inverse kinematics, the precurved tube needed to be outstretched by 45 mm and 70 mm for the upper and lower target positions, respectively. The precurvature of the assembly including the precurved tube, inner cutting and suction tubes was measured right away after they were assembled and found to be about 7.2 m−1. After accessing each target position, the precurved tube was retracted into the outer tube, rotated, and then extended to access the next target position as needed. To measure the positions of the robot tip and compare them with the target positions, a depth camera (ZED 2i Stereo Camera, StereoLabs, France) was used. A modified version of the robot without the cutting wires but instead with a red foam ball marker at the conic tip of the robot was used for this study, as shown in FIG. 13A. In addition, a 3D-printed frame with three green foam ball markers was mounted on the outer tube to provide a reference coordinate by the camera. FIG. 13B shows a series of snapshots when the robot moves to the desired target positions for the maximum length. FIG. 14 shows the measured positions of the robot tip along with the target positions. The average tip position error for the upper and lower target positions was 2.3±0.4 mm and 3.1±1.3 mm, respectively, corresponding to 5.1±0.9% and 4.4±1.9% of the extended length of the precurved tube, respectively. This error can be attributed to the assumption of constant curvature which may not be accurate due to the shape setting of large-diameter notched tube. This error can be compensated by designing a small safety margin, adjusting the cutting wire expansion extent, and manipulating the device under intra-operative imaging guidance, such as magnetic resonance imaging (MRI).

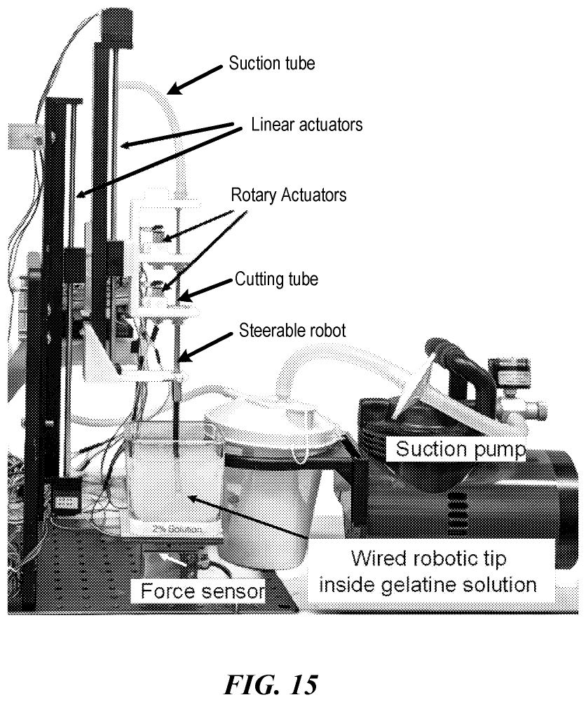

To demonstrate the ability of some example robots to remove brain mass lesions, an experimental setup was developed, as shown in FIG. 15. The experimental setup includes an example robotic system (e.g., system 1100) vertically placed above a glass container filled with gelatin. The force sensor and suction machine were not used in the demonstration. The inner polymer tube of the robot was connected to an aspiration machine. The gelatin phantom was made by mixing gelatin powder with water in 2% mass ratio. The modulus of 2% gelatin was measured to be about 1.9 kPa using an indentation test. Thus, the stiffness of the gelatin phantom was in the range of normal brain tissue and close to glioma and metastatic lymphoma.

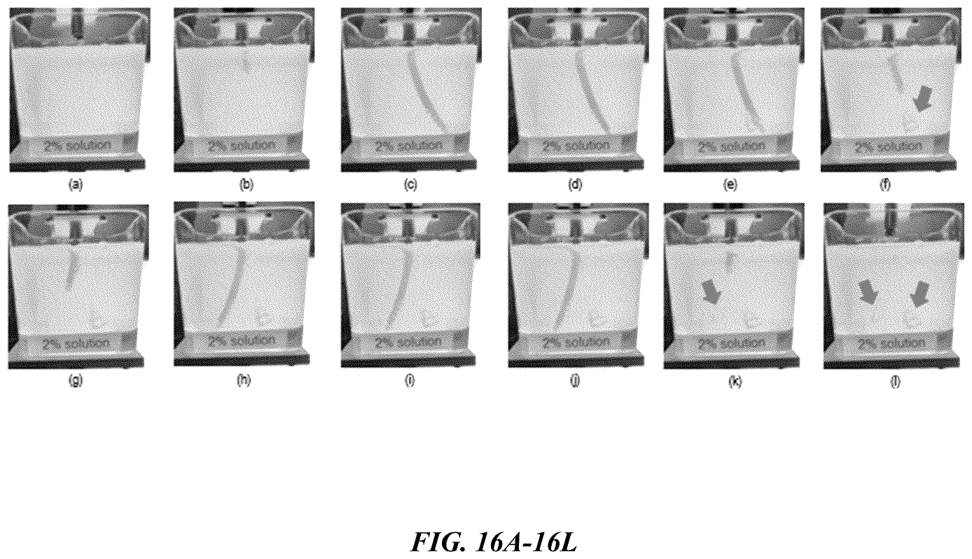

FIGS. 16A-16L shows example images demonstrating tissue removal in the gelatine phantom using the robotic system in the experiment of FIG. 15. Specifically, FIG. 16A shows the steerable robot with the precurved tube and cutting wires inside the outer tube, FIG. 16B shows inserting the straight robot into gelatine, FIG. 16C shows advancing the robot along a curved trajectory by extending the precurved tube, FIG. 16D shows robot tip with unwound, expanded cutting wires, FIG. 16E shows winding the cutting wires after tissue removal, FIG. 16F shows retracting the precurved tube towards the outer tube, FIG. 16G shows robot with the precurved tube rotated to a new direction, FIG. 16H shows extending the precurved tube to access a new location, FIG. 16I shows robot tip with unwound, expanded cutting wires at the new location, FIG. 16J shows winding the cutting wires after tissue removal, FIG. 16K shows retracting the precurved tube into the outer tube, and FIG. 16L shows retracting the steerable robot out of the gelatine phantom. The red arrows indicate the locations of generated cavities.

During the experiment, before inserting the robot into the gelatin phantom, the robot was set in the initial state by fully retracting the precurved tube into the outer tube, as shown in FIG. 16A. The robot was then inserted into the gelatin phantom by extending all tubes concurrently and manipulated to advance along a curved trajectory by extending the precurved tube and polymer tubes by 70.0 mm, as shown in FIG. 16B and FIG. 16C. At the deep location in the gelatin phantom, the outer polymer tube was rotated back and forth in the range of 220° to fully unwind and wind the cutting wires for 20 cycles at the speed of 2.8 seconds per cycle, as shown in FIG. 16D. The aspiration machine was operated at 6.24 PSI from 10 seconds before to 10 seconds after the operation of the cutting wires. FIG. 16E and FIG. 16E. show the generation of a cavity at the end of aspiration and during the retraction of the precurved tube towards the outer tube, respectively. By rotating the precurved tube by 150°, the above procedure was repeated to create another cavity, as shown in FIGS. 16G-16K. Finally, the precurved tube was fully retracted into the outer tube, and the robot was removed from the gelatin phantom, as shown in FIG. 16L.

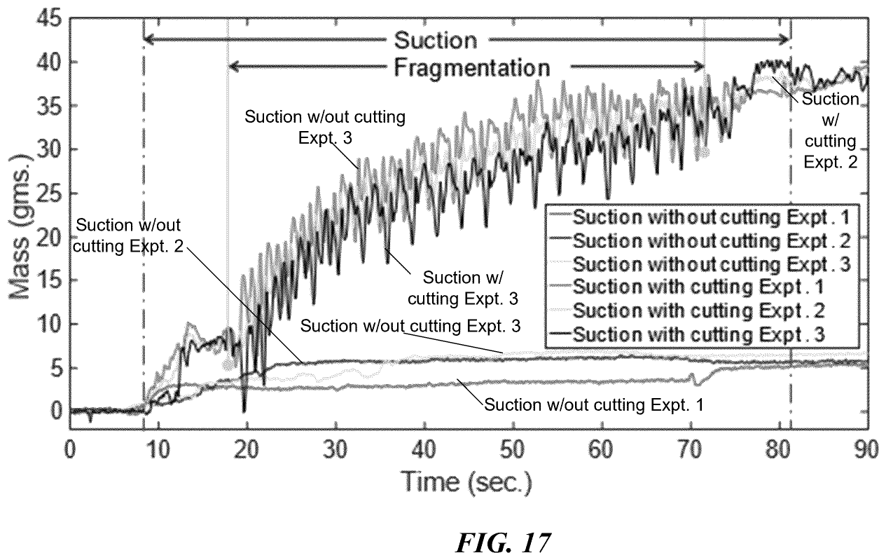

To evaluate the efficiency of the robotic system in tissue removal, comparison was made between case i—the robot with the operation of both cutting wires and aspiration and case ii—the same robot with only aspiration. The experimental setup is shown in FIG. 15, with a load cell (Omega Engineering Inc., USA) placed under the phantom container to measure the change in weight of the gelatin phantom. Each case was repeated three times. Each test was performed using a new 2% gelatin phantom to minimize the interference between the tests. In case i, the tissue cutting and aspiration mechanism was operated in the same manner as the demonstration. In case ii, the cutting wires were removed, and aspiration was applied for the same amount of time. FIG. 17 shows the change of the phantom mass for case i. The experimental results of FIG. 17 show that in case i, about 37.8±0.3 g gelatin tissue was removed. In case ii, only 5.9±0.6 g gelatin tissue was removed and tissue removal stopped in about 8.5±5.4 s after aspiration, since the aspiration apertures and polymer tubes were clogged by the chucks of tissue. Thus, some disclosed devices with capabilities of both fragmentation an aspiration can improve the efficiency and efficacy of tissue removal by about 540.68% compared to aspiration only. Compared to FDA-approved straight debriders, such as NICO Myriad®, the efficiency of some disclosed embodiments is one order of magnitude higher.

The present patent document discloses, among other things, a mesoscale steerable neurosurgical robot that can be implemented for minimally invasive debulking of brain mass lesions. The robot has the ability to navigate a mass lesion and remove tissue at multiple locations. In some embodiments, the ability of efficient tissue removal without clogging through a single insertion can reduce collateral damage, surgical time, and potentially complications. In some embodiments, this ability is enabled by the novel design of the robot, which includes a straight rigid outer tube, a precurved superelastic nitinol tube, an outer braid-reinforced polymer tube with aspiration apertures, an inner braid-reinforced polymer tube connected to a suction machine, and four superelastic wire loops between the tips of the outer polymer tube and precurved tube. By rotating the outer polymer tube, tissue enveloped by the wire loops can be fragmented, drawn in the rotary aspiration apertures, dissected by the aperture edges, and removed through the inner polymer tube. The precurved nitinol tube can be machined with notches to allow for shape setting and translation inside the straight tube. By rotating and translating the outer tube and precurved tube, the robot tip with the tissue cutting and aspiration mechanism can be manipulated. Results obtained in an example experimental study show that an example robot can achieve positioning accuracy of about 4% to 5% of the extended length of the precurved tube in air and improve the tissue removal efficiency by 540% compared to pure suction.

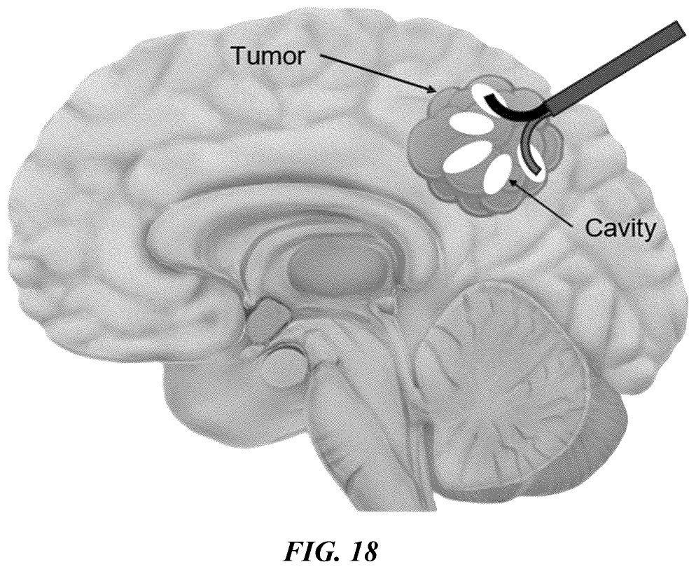

FIG. 18 shows a schematic of an example implementation of a steerable robotic device according to an embodiment of the disclosed technology. Removal of intracranial mass legions at multiple locations can be achieved using a steerable robotic device equipped with a tissue cutting mechanism in accordance with some disclosed embodiments.

The present patent document discloses, among other things, a robotic surgical device for minimally invasive removal of brain mass lesions. Compared to existing steerable robots and conventional tools, some disclosed embodiments enable more effective removal of brain mass lesions by manipulating a tissue fragmentation and aspiration system inside a mass lesion, thus allowing tissue removal at multiple locations under imaging guidance. Some disclosed embodiments relate to creation of a CTR robot with a sufficiently large channel. Some disclosed embodiments relate to the implementation and integration of the tissue fragmentation and aspiration mechanism with a CTR hosting a sufficiently large channel. In some embodiments, the large-diameter CTR design can avoid clogging during tissue removal and can be achieved by applying notch patterns to reduce stiffness prior to shape setting. Compared to similar designs that focus on improving the stability of small-diameter CTRs, some disclosed embodiments can be implemented to overcome the strain limit and achieve a larger internal channel.

Other examples of surgical devices and methods that may be implemented in accordance with the present technology are described in U.S. Patent Publication No. 9,320,540 B2 (“Surgical Devices and Related Methods Thereof”), U.S. Patent Publication No. 10,702,336 B2 (“Surgical Devices”), and U.S. Patent Publication No. 11,259,865 B2 (“Surgical Methods”), which are incorporated by reference as part of the disclosure of this patent document for all purposes.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed:

1. A steerable apparatus for modular debriding, comprising:
a shaft comprising a central lumen that extends along a length of the shaft;
an adapter coupled to a proximal end of the shaft;
at least one bearing comprising a central opening and positioned along the shaft such that the central opening is concentric with the central lumen and the adapter;
at least one disk coupled to the shaft or to the at least one bearing;
a pair of shape memory alloy (SMA) springs, the at least one disk or the at least one bearing being positioned along the shaft and between the pair of SMA springs such that the pair of SMA springs are spaced apart from one another along the length of the shaft by the at least one disk or the at least one bearing;
a housing comprising (i) a top cover positioned at a top surface of the housing and (ii) a bottom cover positioned at a bottom surface of the housing, wherein the shaft, the central lumen, the at least one bearing, and the pair of SMA springs are disposed within the housing;
a tip structure coupled to a distal end of the housing; and
rotatable SMA cutting wire loops disposed on an external surface of the housing and coupled to the top cover and the tip structure.

2. The steerable apparatus of claim 1, wherein the adapter is coupled to a distal end of a surgical tool, wherein the surgical tool comprises:
a first flexible tube coupled to the housing and configured to curve at the distal end of the surgical tool such that the rotatable SMA cutting wire loops are steerable in a direction based on curvature of the first flexible tube.

3. The steerable apparatus of claim 2, wherein the first flexible tube is further configured to translate such that the rotatable SMA cutting wire loops are steerable based on at least one of translation or curvature of the first flexible tube.

4. The steerable apparatus of claim 2, wherein the surgical tool further comprises:
a rigid tube; and
a second flexible tube, wherein a distal portion of the second flexible tube comprises notches disposed over a distal portion of the first flexible tube,
wherein the second flexible tube and the first flexible tube are disposed within the rigid tube.

5. The steerable apparatus of claim 2, wherein the surgical tool, with the steerable apparatus, are robotically operated to debride a mass from a human subject.

6. The steerable apparatus of claim 1, wherein the adapter includes a suction channel and one or more irrigation channels, wherein mass fragmented by the rotatable SMA cutting wire loops is debrided via the suction channel.

7. The steerable apparatus of claim 1, wherein the steerable apparatus is coupled to a surgical tool configured to navigate the steerable apparatus while the steerable apparatus is located inside a mass.

8. The steerable apparatus of claim 7, wherein navigation of the steerable apparatus while the steerable apparatus is located inside the mass includes curving and translation of the steerable apparatus.

9. A steerable apparatus for modular debriding, comprising:
a rigid tube;
a precurved flexible tube disposed within the rigid tube and advanceable beyond a distal end of the rigid tube;
a first flexible tube disposed within the precurved flexible tube and the rigid tube, a distal end of the first flexible tube extendable beyond a distal end of the precurved flexible tube;
a second flexible tube disposed within the first flexible tube, the precurved flexible tube, and the rigid tube, a distal end of the second flexible tube extendable beyond the distal end of the precurved flexible tube and the distal end of the first flexible tube; and
a tip structure coupled to the distal end of the second flexible tube and comprising rotatable shape memory alloy (SMA) cutting wire loops, each of the rotatable SMA cutting wire loops disposed between a distal portion of the tip structure and an exterior surface of at least one of the distal end of the first flexible tube,
wherein the rigid tube, the precurved flexible tube, the first flexible tube, and the second flexible tube are coupled to one another sₒ as to form a steerable tool operable to navigate the tip structure based on a direction of the precurved flexible tube and while the tip structure is located inside a mass,
wherein the direction of the precurved flexible tube is based on translating the rigid tube and translating and rotating the precurved flexible tube,
wherein the rotatable SMA cutting wire loops are configured to twist, expand, and rotate based on rotation of respective proximal ends of the first flexible tube and the second flexible tube with respect to one another.

10. The steerable apparatus of claim 9, wherein the tip structure is configured to navigate linearly or curvilinearly based on the direction of the precurved flexible tube and while the tip structure is located inside the mass.

11. The steerable apparatus of claim 9, wherein the rotatable SMA cutting wire loops are configured to rotate via rotation of the first flexible tube and the second flexible tube.

12. The steerable apparatus of claim 9, further comprising electrical wires disposed between the precurved flexible tube and the first flexible tube, wherein the electrical wires are configured to pass electric current to the rotatable SMA cutting wire loops such that the rotatable SMA cutting wire loops are capable of electrocauterization.

13. The steerable apparatus of claim 9, wherein the steerable apparatus is robotically operated.

14. The steerable apparatus of claim 9, wherein the first flexible tube and the second flexible tube each include a channel, wherein the mass is aspirated through the channel of at least one of the first flexible tube or the second flexible tube.

15. The steerable apparatus of claim 9, wherein the tip structure further comprises:
a shaft comprising a central lumen that extends along a length of the shaft;
an adapter coupled to a proximal end of the shaft;
at least one bearing comprising a central opening and positioned along the shaft such that the central opening is concentric with the central lumen and the adapter;
at least one disk coupled to the shaft or to the at least one bearing;
a pair of shape memory alloy (SMA) springs, the at least one disk or the at least one bearing being positioned along the shaft and between the pair of SMA springs such that the pair of SMA springs are spaced apart from one another along the length of the shaft by the at least one disk or the at least one bearing; and
a housing comprising (i) a top cover positioned at a top surface of the housing and (ii) a bottom cover positioned at a bottom surface of the housing, wherein the shaft, the central lumen, the at least one bearing, and the pair of SMA springs are disposed within the housing.

16. The steerable apparatus of claim 9, wherein the first flexible tube and the second flexible tube are configured to bend in the direction of the precurved flexible tube while the tip structure is located inside the mass.

17. The steerable apparatus of claim 9, wherein the rotatable SMA cutting wire loops are actuated to fragment the mass, wherein a distal portion of the precurved flexible tube includes notches through which suction is applied to aspirate the fragmented mass.

18. A steerable apparatus for modular debriding, comprising:
a rigid tube;
a tube comprising one or more notches located at a distal end of the tube;
a straight flexible tube, disposed within the rigid tube and the tube, comprising a proximal end, wherein the straight flexible tube includes along its length one or more openings configured to align with the one or more notches;
a tip structure located at the distal end of the tube and configured to cap the straight flexible tube at one end; and
metallic wires forming loops disposed between the tip structure and the tube,
wherein the rigid tube, the tube, and the straight flexible tube are coupled to one another so as to form a steerable tool operable to navigate the tip structure,
wherein rotation of the proximal end allows the metallic wires to be twisted and expanded.

19. The steerable apparatus of claim 18, wherein at least one superelastic SMA wire is attached to the tip structure, wherein the at least one superelastic SMA wire is routed through the straight flexible tube to actuators located external to the steerable apparatus.

20. The steerable apparatus of claim 19, wherein the tube is a precurved flexible tube, wherein translation of the rigid tube and the precurved flexible tube allows the tip structure and the metallic wires to navigate.
